(19) United States
(12) Patent Application Publication
(10) Pub. No.: US 2019/0365760 A1
(43) Pub. Date: Dec. 5, 2019

(54) A THERAPEUTIC OR PROPHYLACTIC AGENT FOR ISCHEMIC DISEASE, GLAUCOMA, OPTIC NERVE DISEASE, RETINAL DEGENERATIVE DISEASE, ANGIOGENIC RETINAL DISEASE, CANCER, NEURODEGENERATIVE OR AUTOIMMUNE DISEASE, AND A HYPOXIA INDUCING FACTOR INHIBITOR

(71) Applicant: KEIO UNIVERSITY, Tokyo (JP)

(72) Inventors: Toshihide KURIHARA, Tokyo (JP); Yukihiro MIWA, Tokyo (JP); Kazuo TSUBOTA, Tokyo (JP); Hiroyoshi INOUE, Tokyo (JP)

(21) Appl. No.: 16/349,821

(22) PCT Filed: Nov. 14, 2017
(86) PCT No.: PCT/JP2017/040884

§ 371 Date: May 14, 2019

(30) Foreign Application Priority Data
Nov. 14, 2016 (JP) 2016-221547

(51) Int. Cl. A61K 31/517; A61K 36/185; A61K 36/9066; A61K 35/60; A61P 27/02

(52) U.S. Cl. CPC A61K 31/517; A61K 36/185; A61P 27/02; A61K 35/60; A61K 36/9066

(57) ABSTRACT

Novel HIF inhibitors having low cytotoxicity and suitable for ophthalmologic treatment and agents for the treatment or prevention of ischemic diseases, glaucoma, optic nerve diseases, retinal degenerative diseases, retinal degenerative diseases, angiogenic retinal diseases, cancer, neurodegenerative or autoimmune diseases are provided.

The present invention is a therapeutic or prophylactic agent for ischemic disease, glaucoma, optic nerve disease, retinal degenerative disease, retinal degenerative disease, angiogenic retinal disease, cancer, neurodegenerative disease, or autoimmune disease, which comprises as an active ingredient one or more or an extract thereof selected from the group consisting of plants belonging to the genus Asian, plants belonging to the genus Swiss, plants belonging to the genus Grape, plants belonging to the genus Brassica, fish belonging to the genus Kivinago, and so on.

### TECHNICAL FIELD

The present invention relates to agents for the treatment or prevention of ischemic diseases, glaucoma, optic nerve diseases such as ischemic optic neuropathy, traumatic optic neuropathy, optic neuritis, retinal degenerative diseases, angiogenic retinal diseases, cancer, neurodegenerative or autoimmune diseases, and inhibitors of hypoxia inducing factors.

### BACKGROUND ART

HIF (Hypoxia Inducible Factor: Hypoxia Inducing Factor) is a transcription factor induced when the oxygen supply to the cell becomes insufficient, and has various physiological activities such as hypoxia adaptive response, maintenance of stem cells, homeostasis maintenance of inflammation, etc., and on the other hand, it is considered that the overexpression of HIF is largely related to the development and progress of cancer.

Therefore, for example, as disclosed in Non-Patent Document 1, HIF has been studied as a target of cancer treatment.

### PRIOR ART DOCUMENTS

### Non-Patent Documents

Non-patent Document 1: Onnis B, et al. “Development of HIF-1 inhibitors for cancer therapy” J Cell Mol Med. 2009,

### SUMMARY OF THE INVENTION

### Problems to be Solved by the Invention

On the other hand, not only cancer but also diseases in the ophthalmologic field can be expected to have a therapeutic effect by inhibiting HIF. However, since many of the HIF inhibitors are anticancer agents as disclosed in Non-Patent Document 1, they have strong cytotoxicity, and therefore, it is difficult to apply the HIF inhibitors as they are to eye diseases, for example. Therefore, new HIF inhibitors with high safety are required.

Many ocular diseases are associated with retinal ischemia, and such ocular diseases include retinal artery occlusion, retinal venous occlusion, diabetic retinopathy, glaucoma, age-related macular degeneration, and the like. These ocular diseases directly cause vision loss and may cause irreversible impairment of visual function. Currently, anti VEGF (Vascular Endothelial Growth Factor) antibody-based therapies have been clinically applied to the complications of retinal ischemic diseases, but long-term anti VEGF therapy has been suggested to cause retinal tissue damage and to affect vision.

The present invention has been made in view of the above circumstances, and an object thereof is to provide a novel HIF inhibitor having low cytotoxicity and suitable for ophthalmologic treatment, and a therapeutic or preventive agent for ischemic disease, glaucoma, optic nerve disease, retinal degenerative disease, angiogenic retinal disease, cancer, neurodegenerative disease, or autoimmune disease.

### Means for Solving the Problems

The present inventors have found that certain plants and fish have high safety and HIF inhibitory activity, and that HIF, which is a transcription factor of VEGF, can be a new therapeutic target to replace VEGF therapy against retinal ischemic diseases, and have completed the present invention. More specifically, the present invention has the following configuration.

(1) A therapeutic or prophylactic agent for ischemic disease, glaucoma, optic nerve disease, retinal pigment degeneration, angiogenic retinal disease, cancer, neurodegeneration, or autoimmune disease, comprising as an active ingredient one or more or an extract thereof selected from plants belonging to the genus Geranium, plants belonging to the genus Morelia, plants belonging to the genus Narcissus, plants belonging to the genus Hydrangea, plants belonging to the genus Vitis, plants belonging to the genus Salix, plants belonging to the genus Camellia, plants belonging to the genus Urtica, plants belonging to the genus Cleyera, plants belonging to the genus Raphanus, plants belonging to the genus Peumus, plants belonging to the genus Passiflora, plants belonging to the genus Cryptomeria, plants belonging to the genus Tulipa, plants belonging to the genus Panax, plants belonging to the genus Curcuma, plants belonging to the genus Brassica, plants belonging to the genus Echinocactus, plants belonging to the genus Tabebuia, plants belonging to the genus Dioscorea, plants belonging to the genus Prunus, plants belonging to the genus Wolfiporia, plants belonging to the genus Amygdalus, plants belonging to the genus Spinacia, plants belonging to the genus Capsicum, plants belonging to the genus Mangifera, plants belonging to the genus Saxifraga, plants belonging to the genus Colocasia, plants belonging to the genus Ulva, plants belonging to the genus Sansevieria, plants belonging to the genus Benincasa, plants belonging to the genus Zanthoxylum, plants belonging to the genus Glycine, fishes belonging to the genus Spratelloides, and fishes belonging to the genus Hyperoglyphe.

(2) A therapeutic or prophylactic agent according to (1), comprising as an active ingredient one or more or an extract thereof selected from the group consisting of plants belonging to a genus of Hydrangea, plants belonging to a genus of Narcissus, plants belonging to a genus of Vitis, and fishes belonging to a genus of Spratelloides.

(3) A therapeutic or prophylactic agent according to (1), comprising as an active ingredient one or more or an extract thereof selected from the group consisting of Hydrangea macrophylla, Narcissus tazetta, Vitis vinifera, B. oleracea var. italica, and Spratelloides gracilis.

(4) A hypoxia inducing factor inhibitor comprising one or more or an extract thereof selected from plants belonging to the genus Geranium, plants belonging to the genus Morelia, plants belonging to the genus Narcissus, plants belonging to the genus Hydrangea, plants belonging to the genus Vitis, plants belonging to the genus Salix, plants belonging to the genus Camellia, plants belonging to the genus Urtica, plants belonging to the genus Cleyera, plants belonging to the genus Raphanus, plants belonging to the genus Peumus, plants belonging to the genus Passiflora, plants belonging to the genus Cryptomeria, plants belonging to the genus Tulipa, plants belonging to the genus Panax, plants belonging to the genus Curcuma, plants belonging to the genus Brassica, plants belonging to the genus Echinocactus, plants belonging to the genus Tabebuia, plants belonging to the genus Dioscorea, plants belonging to the genus Amygdalus, plants belonging to the genus Wolfiporia, plants belonging to the genus Amygdalus, plants belonging to the genus Spinacia, plants belonging to the genus Capsicum, plants belonging to the genus Mangifera, plants belonging to the genus Saxifraga, plants belonging to the genus Colocasia, plants belonging to the genus Ulva, plants belonging to the genus Sansevieria, plants belonging to the genus Benincasa, plants belonging to the genus Zanthoxylum, plants belonging to the genus Glycine, fishes belonging to the genus Spratelloides, and fishes belonging to the genus Hyperoglyphe.

(5) The hypoxia-inducing factor inhibitor according to (4), comprising one or more or an extract thereof selected from the group consisting of plants belonging to the genus Hydrangea, plants belonging to the genus Narcissus, plants belonging to the genus Vitis, plants gelonging to the genus Brassica, and fishes belonging to the genus Spratelloides.

(6) The hypoxia-inducing factor inhibitor according to (4), comprising one or more or an extract thereof selected from the group consisting of Hydrangea macrophylla, Narcissus tazetta, Vitis vinifera, B. oleracea var. italica, and Spratelloides gracilis.

(7) A retinal neuroprotective agent comprising the hypoxia inducing factor inhibitor according to any one of (4) to (6) above.

(8) A therapeutic or prophylactic agent for ischemic disease, glaucoma, optic nerve disease, retinal pigment degeneration, or neurodegeneration, comprising halofuginone as an active ingredient.

(9) Hypoxia inducing factor inhibitor comprising halofuginone.

(10) A retinal neuroprotective agent comprising halofuginone.

### Effect of the Invention

According to the present invention, the HIF inhibiting effect can be achieved while the cytotoxicity is kept low. The present invention may also provide a therapeutic or prophylactic agent for ischemic disease, glaucoma, optic nerve disease, retinal pigment degeneration, angiogenic retinal disease, cancer, neurodegeneration, or autoimmune disease, which may protect retinal nerves.

### BRIEF DESCRIPTION OF THE DRAWINGS

FIG. 1 is a graph of HIF activity measured in 661W cells using CoCl₂ and topo or DXR.

FIG. 2 is a graph of HIF activity measured in NIH3T3 cells using CoCl₂ and topo or DXR.

FIG. 3 is a graph of HIF activity measured in 661W cells using CoCl₂ and extracts of Narcissus bulbs, Narcissus roots, Hydrangea macrophylla flowers ⋅ sepals or Narcissus leaves.

FIG. 4 is a graph of HIF activity measured in 661W cells using CoCl₂ and extracts of Vitis branches, or Spratelloides gracilis.

FIG. 5 is a graph of HIF activity measured in NIH3T3 cells using CoCl₂ and extracts of Narcissus leaves, Hydrangea macrophylla flowers ⋅ sepals, Narcissus bulbs, Narcissus roots, Vitis branches, Salix gracilistyla, Spratelloides gracilis, Camellia pistils ⋅ stamens, Camellia flowers (white), Urtica, Raphanus leaves, Cleyera branches, Raphanus roots, Peumus boldus, Passiflora caerulea, Cryptomeria japonica, Tulipa leaves, Panax ginseng, Camellia flowers (pink), and Sansevieria trifasciata.

FIG. 6 is a graph of HIF activity measured in 661W cells using CoCl₂ and extracts of Hydrangea macrophylla stems.

FIG. 7 is a graph of HIF activity measured in NIH3T3 cells using CoCl₂ and extracts of Hydrangea macrophylla stems, Camellia japonica (Koiso) calyxes, Camellia japonica (Benishosuke) calyxes, Geranium thunbergii roots, Curcuma longa powder, Tabebuia avellanedae tea, Benincasa hispida fruits ⋅ seeds, Geranium thunbergii roots, Wolfiporia extensa, B. oleracea var. italica roots, Urtica, Spinacia leaves, Colocasia leaves, Hyperroglyphe japonica, Zanthoxylum piperitum or Namupuri-pepper.

FIG. 8 is a graph of HIF activity measured in 661W cells using CoCl₂ and extracts of Hydrangea macrophylla flowers ⋅ sepals, or B. oleracea var. italica.

FIG. 9 is a graph of HIF activity measured in NIH3T3 cells using CoCl₂ and extracts of Hydrangea macrophylla flowers ⋅ sepals, GROWTH FACTOR CONCENTRATE, Fish Protein, Curcuma longa powder, Boniron, tea branches, Bio-Active Shark Cartilage Powder, B. oleracea var. italica, Echinocactus roots, Hyperroglyphe japonica, Brassica rapa, Tahibo tea, Propagule, Mangifera seeds, Prunus seeds, Saxifraga roots, Wolfiporia extensa, green soybeans peel, almond shell, Colocasia leaves, or Ulva.

FIG. 10 is a graph of HIF activity measured in NIH3T3 cells using CoCl₂ and extracts of Geranium roots or Morella leaves.

FIG. 11 is a graph of HIF activity measured in 661W cells using CoCl₂ and halofuginone.

FIG. 12 is a graph of HIF activity measured in NIH3T3 cells using CoCl₂ and halofuginone.

FIG. 13 is a graph of HIF activity measured in ARPE19 cells using CoCl₂ and halofuginone.

FIG. 14 is a graph of 661W cells in which (a) the expression amount of the bnip3 gene, (b) the expression amount of the glut1 gene, and (c) the expression amount of the pdk1 gene were measured using CoCl₂ and halofuginone.

FIG. 15 is a graph of HIF activity measured in NIH3T3 cells under hypoxic conditions using Spratelloides gracilis.

FIG. 16 shows the electroretinogram (rod and cone functional evaluation) performed in the dark of the halofuginone group and the PBS group (control).

FIG. 17 shows the electroretinogram (cone function evaluation) performed in the light of the halofuginone group and the PBS group.

FIG. 18 shows the electroretinogram (ERG) measurements in the halofuginone and PBS groups.

FIG. 19 shows the results of visual evoked potential (VEP) measurements in the halofuginone and PBS groups.

FIG. 20 is a graph showing measurement results of total retinal thickness by optical coherence tomography (OCT) in the halofuginone group and the PBS group.

### MODE FOR CARRYING OUT THE INVENTION

Although specific embodiments of the present invention will be described in detail below, the present invention is not limited to the following embodiments, and can be implemented with appropriate modifications within the scope of the object of the present invention.

Descriptions of overlapping portions may be omitted as appropriate, but the gist of the invention is not limited.

<Therapeutic or Preventive Agent>

The therapeutic or prophylactic agent of the present invention is a therapeutic or prophylactic agent for ischemic diseases, glaucoma, optic nerve disease, retinal pigment degeneration, angiogenic retinal disease, cancer, neurodegeneration, or autoimmune disease, wherein the therapeutic or prophylactic agent is plants belonging to the genus Geranium, plants belonging to the genus Morelia, plants belonging to the genus Narcissus, plants belonging to the genus Hydrangea, plants belonging to the genus Vitis, plants belonging to the genus Salix, plants belonging to the genus Camellia, plants belonging to the genus Urtica, plants belonging to the genus Cleyera, plants belonging to the genus Raphanus, plants belonging to the genus Peumus, plants belonging to the genus Passiflora, plants belonging to the genus Cryptomeria, plants belonging to the genus Tulipa, plants belonging to the genus Panax, plants belonging to the genus Curcuma, plants belonging to the genus Brassica, plants belonging to the genus Echinocactus, plants belonging to the genus Tabebuia, plants belonging to the genus Dioscorea, plants belonging to the genus Prunus, plants belonging to the genus Wolfiporia, plants belonging to the genus Amygdalus, plants belonging to the genus Spinacia, plants belonging to the genus Capsicum, plants belonging to the genus Mangifera, plants belonging to the genus Saxifraga, plants belonging to the genus Colocasia, plants belonging to the genus Ulva, plants belonging to the genus Sansevieria, plants belonging to the genus Benincasa, plants belonging to the genus Zanthoxylum, plants belonging to the genus Glycine, fishes belonging to the genus Spratelloides, and fishes belonging to the genus Hyperoglyphe, or extracts thereof.

As will be described later, the above-mentioned plants and fish have HIF inhibitory activity. By exerting HIF inhibitory activity or protecting retinal nerves, ischemic diseases, glaucoma, optic nerve diseases (ischemic optic neuropathy, traumatic optic neuropathy, optic neuritis, etc.), retinal degenerative diseases, angiogenic retinal diseases, cancer, neurodegeneration, or autoimmune diseases can be treated or prevented. In particular, the therapeutic or prophylactic agents of the present invention are useful because they are not cytotoxic. The therapeutic agent or prophylactic agent of the present invention is useful, for example, for diseases in the ophthalmologic field, such as ischemic diseases, glaucoma, optic nerve diseases, cancer of the eye, retinal degenerative diseases, angiogenic retinal diseases, and the like.

Plants belonging to the genus Geranium which can be included in the therapeutic or prophylactic agent of the present invention are not particularly limited, and examples thereof include G. thunbergii, G. sibiricum, G. tripartitum, G. wilfordii, G. yesoemse, G. shikokianum, G. soboliferum, G. krameri, G. yoshinoi, G. eriostemon, G. erianthum, G. carolinianum, G. robertianum, and G. molle, and the like. Of these, Geranium thunbergii is particularly preferable.

Plants belonging to the genus Morelia which can be included in the therapeutic or prophylactic agent of the present invention are not particularly limited, and examples thereof include M. rubra, M. adenophora, M. cerifera, and the like. Among these, M. rubra is particularly preferable.

Plants belonging to the genus Narcissus which can be included in the therapeutic or prophylactic agent of the present invention are not particularly limited, and examples thereof include N. tazetta, N. tazetta var. chinensis, N. pseudonarcissus, N. poeticus, N. jonquilla, N. odorus, and the like. Of these, N. tazetta is particularly preferable.

Plants belonging to the genus Hydrangea which can be included in the therapeutic or prophylactic agents of the present invention are not particularly limited, and examples thereof include H. macrophylla, H. macrophylla f. normalis, H. serrata, H. stylosa, H. serrata var. megacarpa, H. serrata f. prolifera, and the like. Among these, H. macrophylla is particularly preferable.

Plants belonging to the genus Vitis which can be included in the therapeutic or prophylactic agents of the present invention are not particularly limited, and examples thereof include V. coignetiae, V. vinifera, V. labrusea, V. rotundifolia, V. amurensis, V. ficifolia, and the like. Also, the plants belonging to the genus Vitis may be grapes of any variety, such as cardinal, kosyu, kyohou, huziminori, shigyoku, shien, pione, and the like.

Plants belonging to the genus Salix which can be included in the therapeutic or prophylactic agents of the present invention are not particularly limited, and examples thereof include S. gracilistyla, S. acutifolia, S. aegyptiaca, S. alba, S. amygdaloides, S. aquilonia, and the like. Of these, S. gracilistyla are particularly preferable.

Plants belonging to the genus Camellia which can be included in the therapeutic or prophylactic agent of the present invention are not particularly limited, and examples thereof include C. japonica, C. lutchuensis, C. rusticana, C. sasanqua, C. sinensis, C. assimilis, and the like. Of these, C. japonica and C. sinensis are particularly preferable.

Plants belonging to the genus Urtica which can be included in the therapeutic or prophylactic agent of the present invention are not particularly limited, and examples thereof include U. thunbergiana, U. dioica, U. Fischer, U. laetevirens, U. platyphylla, U. urens, and the like. Of these, U. thunbergiana is particularly preferable.

Plants belonging to the genus Cleyera which can be included in the therapeutic or prophylactic agent of the present invention are not particularly limited, and examples thereof include C. japonica, C. ochnacea, C. millettii, C. integrifolia, and the like. Of these, C. japonica is particularly preferable.

Plants belonging to the genus Raphanus which can be included in the therapeutic or prophylactic agent of the present invention are not particularly limited, and examples thereof include R. sativus, R. sativus var. sativus, R. sativus var. L. var. raphanistroides, R. sativus var. longipinnatus, and the like. Of these, R. sativus var. sativus is particularly preferable.

Plants belonging to the genus Peumus which can be included in the therapeutic or prophylactic agent of the present invention are not particularly limited, and examples thereof include P. boldus and the like, with P. boldus being preferred.

Plants belonging to the genus Passiflora which can be included in the therapeutic or prophylactic agent of the present invention are not particularly limited, and examples thereof include P. caerulea, P. coccinea, P. foetida, P. incarnata, P. ligularis, P. quadrangularis, P. racemosa, and the like. Of these, P. caerulea is particularly preferred.

Plants belonging to the genus Cryptomeria which can be included in the therapeutic or prophylactic agent of the present invention are preferably C. japonica.

Plants belonging to the genus Tulipa which can be included in the therapeutic or prophylactic agent of the present invention are not particularly limited, and examples thereof include T. gesneriana, T. kaufmaniana, T. fosteriana, T. saxatilis, T. praestans, T. greigii, T. linifolia, and the like.

Plants belonging to the genus Panax which can be included in the therapeutic or prophylactic agent of the present invention are not particularly limited, and examples thereof include P. ginseng, P. notoginseng, P. bipinnatifidus, P. japonicus, P. quinquefolius, P. vietnamensis, P. pseudoginseng, and the like. Of these, P. ginsen is particularly preferable.

Plants belonging to the genus Curcuma which can be included in the therapeutic or prophylactic agent of the present invention are not particularly limited, and examples thereof include C. longa, C. amada, C. aromatica, C. kwangsinensis, C. mangga, C. angustifolia, and the like. Of these, C. longa is particularly preferable.

Plants belonging to the genus Brassica which can be included in the therapeutic or prophylactic agent of the present invention are not particularly limited, and examples thereof include B. oleracea var. italica, B. oleracea var. botrytis, B. juncea var. integrifolia, B. juncea var. tumida, B. rapa var. laciniifolia, B. rapa subsp. rapa, B. rapa var. hakabura, B. rapa var. perviridis, B. rapa subsp. pekinensis, B. rapa subsp. chinensis, B. napus, B. carinata, B. rapa var. nipposinica, and the like. Of these, B. oleracea var. italica, B. juncea var. tumida, and B. rapa subsp. rapa, are particularly preferable.

Plants belonging to the genus Echinocactus which can be included in the therapeutic or prophylactic agent of the present invention are not particularly limited, and examples thereof include E. grusonii, E. polycephalus, E. parryi, E. horizonthalonius, and the like. Of these, E. grusonii is particularly preferred.

Plants belonging to the genus Tabebuia which can be included in the therapeutic or prophylactic agent of the present invention are not particularly limited, and examples thereof include T. avellanedae, T. caraiba, T. brevipes, T. cassinoides, T. citrifolia, T. dentata, T. draconcephaloid, T. japurensis, and the like. Of these, T. avellanedae is particularly preferred.

Plants belonging to the genus Dioscorea which can be included in the therapeutic or prophylactic agent of the present invention are not particularly limited, and examples thereof include D. japonica, D. polystachya, D. opposita, D. alata, and the like. Of these, D. japonica is particularly preferable. In addition, the plant belonging to D. japonica can be a propagule.

Plants belonging to the genus Prunus which can be included in the therapeutic or prophylactic agent of the present invention are not particularly limited, and examples thereof include P. domestica, P. salicina, P. mume, P. armeniaca, and the like. Of these, P. domestica is particularly preferable.

Plants belonging to the genus Wolfiporia which can be included in the therapeutic or prophylactic agent of the present invention are not particularly limited, and examples thereof include W. extensa, W. dilatohypha, and the like. Of these, W. extensa is particularly preferable.

Plants belonging to the genus Amygdalus which can be included in the therapeutic or prophylactic agent of the present invention are not particularly limited, and examples thereof include A. dulcis, A. persica, and the like. Of these, A. dulcis are particularly preferred.

Plants belonging to the genus Spinacia which can be included in the therapeutic or prophylactic agent of the present invention are not particularly limited, and examples thereof include S. oleracea, S. tetrandra, S. turkestanica, and the like. Of these, S. oleracea is particularly preferable.

Plants belonging to the genus Capsicum which can be included in the therapeutic or prophylactic agent of the present invention are not particularly limited, and examples thereof include C. annuum, C. frutescens, C. baccatum, and the like. Of these, C. annuum is particularly preferable.

Plants belonging to the genus Mangifera which can be included in the therapeutic or prophylactic agent of the present invention are not particularly limited, and examples thereof include M. indica, M. casturi, M. odorata, and the like. Of these, M. indica is particularly preferable.

Plants belonging to the genus Saxifraga which can be included in the therapeutic or prophylactic agent of the present invention are not particularly limited, and examples thereof include S. stolonifera, S. cernua, S. bracteata, S. bronchialis, S. nipponica, and the like. Of these, S. stolonifera is particularly preferable.

Plants belonging to the genus Colocasia which can be included in the therapeutic or prophylactic agent of the present invention are not particularly limited, and examples thereof include C. antiquorum, C. esculenta, and the like. Of these, C. antiquorum is particularly preferable.

Plants belonging to the genus Ulva which can be included in the therapeutic or prophylactic agent of the present invention are not particularly limited, and examples thereof include U. arasakii, U. clathrata, U. compressa, U. conglobata, U. fenestrata, U. ohnoi, and the like.

Plants belonging to the genus Sansevieria which can be included in the therapeutic or prophylactic agent of the present invention are not particularly limited, and examples thereof include S. trifasciata, S. aethiopica, and the like. Of these, S. trifasciata is particularly preferable.

Plants belonging to the genus Benincasa which can be included in the therapeutic or prophylactic agent of the present invention are not particularly limited, and examples thereof include B. hispida.

Plants belonging to the genus Zanthoxylum which can be included in the therapeutic or prophylactic agent of the present invention are not particularly limited, and examples thereof include Z. piperitum, Z. bungeanum, Z. schinifolium, Z. nitidum, and the like. Of these, Z. piperitum is particularly preferred.

Plants belonging to the genus Glycine which can be included in the therapeutic or prophylactic agent of the present invention are not particularly limited, and examples thereof include G. max, G. soja, and the like. Of these, G. max is particularly preferable.

Plants belonging to the genus Spratelloides which can be included in the therapeutic or prophylactic agent of the present invention are not particularly limited, and examples thereof include S. gracilis, S. atrofasciatus, S. delicatulu, and the like. Of these, S. gracilis is particularly preferred.

Plants belonging to the genus Hyperoglyphe which can be included in the therapeutic or prophylactic agent of the present invention are not particularly limited, and examples thereof include H. japonica, H. antarctica, H. perciformis, and the like. Of these, H. japonica is preferable.

The therapeutic or prophylactic agent of the present invention preferably contains as an active ingredient one or more or an extract thereof selected from the group consisting of a plant belonging to the genus Hydrangea, a plant belonging to the genus Narcissus, a plant belonging to the genus Vitis, a plant belonging to the genus Brassica, and a fish belonging to the genus Spratelloides.

The plant which may be included in the therapeutic or prophylactic agent of the present invention may be any part of the plant, for example, a root, stem, leaf, branch, flower, fruit, seed, etc.

The solvent for extracting the plant or fish extract which can be included in the therapeutic or prophylactic agent of the present invention is not limited, and may be extracted by any solvent such as water, hot water, organic solvent such as alcohol, or mixed solvent of water and organic solvent, for example, but the present invention is useful in that it can be easily extracted by water, hot water, or the like.

For example, in the case where extraction is performed from a plant or fish with water or hot water, extraction may be performed by subjecting an extraction portion of the plant or fish to water, followed by ultrasonic treatment, stirring, centrifugal/supernatant recovery, filtration, or the like. The extract may also be lyophilized.

The therapeutic or prophylactic agent of the present invention may contain halofuginone as an active ingredient. As will be described later, halofuginone is a halogenated derivative of ferbrifudine contained in purple flowers and is used as an antimalarial agent, an anticoccidial agent, an antidiarrheal agent, and the like, but as a result of finding that it has new HIF inhibitory activity and has retinal neuroprotective activity in the present invention, it can be used as a therapeutic or preventive agent for ischemic diseases, glaucoma, optic nerve diseases, retinal degenerative diseases, angiogenic retinal diseases, cancer, neurodegenerative or autoimmune diseases. Halofuginone includes various acid addition salts such as hydrochloride and hydrobromide in addition to the free form, but is not limited to the present invention and any of them can be used. Furthermore, halofuginone has two optically active centers in its molecule, and can be used in any of four optical isomers in addition to racemic isomers, among which [(2R,3S)-3-hydroxy-2-piperidinyl]form is more preferable. Halofuginone can be represented by, for example, the following formula, and can be synthesized according to known literature, or can be purchased as a reagent, an industrial raw material, or the like.

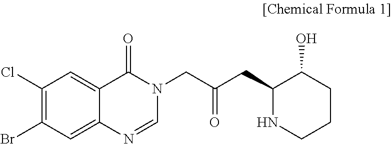 [SMILES: O=C(C[C@@H]1NCCC[C@H]1O)Cn1cnc2cc(Br)c(Cl)cc2c1=O]

The subject disease of the therapeutic or prophylactic agent of the present invention may be any of ischemic disease, glaucoma, optic nerve disease, retinal degenerative disease, angiogenic retinal disease, cancer, neurodegenerative or autoimmune disease, but is suitable for the treatment or prevention of ischemic disease, glaucoma, optic nerve disease, retinal degenerative disease, cancer.

The cancers serving as the target diseases of the therapeutic or prophylactic agent of the present invention are not particularly limited, and include, but are not limited to, eyelid malignancy, horn/conjunctival malignancy, intraocular malignancy, orbital malignancy, lung cancer, prostate cancer, breast cancer, liver cancer, stomach cancer, colorectal cancer, thyroid cancer, kidney cancer, uterine cancer, ovarian cancer, osteosarcoma, chondrosarcoma, rhabdomyosarcoma, leiomyosarcoma, malignant lymphoma, acute/chronic leukemia, myelodysplastic syndrome (MDS), myeloproliferative tumor, and the like.

The retinal degenerative disease which is a target disease of the therapeutic agent or the preventive agent of the present invention is not particularly limited, and includes age-related macular degeneration, retinal pigment degeneration, hereditary macular dystrophy, and the like.

The neurodegeneration which is a target disease of the therapeutic agent or prophylactic agent of the present invention is not particularly limited, but includes amyotrophic lateral sclerosis, Parkinson's syndrome, Alzheimer's disease, progressive supranuclear paralysis, Huntington's disease, multisystem atrophy, spinocerebellar degeneration, and the like.

The angiogenic retinal disease which is a target disease of the present therapeutic agent or prophylactic agent is not particularly limited, and includes retinopathy of prematurity, diabetic retinopathy, proliferative vitreous retinopathy, age-related macular degeneration, VHL (Von Hippel-Lindau) disease, and the like.

Autoimmune diseases which are target diseases of the present therapeutic or prophylactic agent are not particularly limited, and include uveitis, rheumatoid arthritis, systemic lupus erythematosus, antiphospholipid syndrome, polymyositis, dermatomyositis, scleroderma, Sjogren's syndrome, IgG4 related diseases, vasculitis syndrome, mixed connective tissue disease, organ specific autoimmune diseases, and the like.

“Active ingredient” as used herein refers to an ingredient contained in an amount necessary to obtain a therapeutic or prophylactic effect of ischemic disease, glaucoma, optic nerve disease, retinal degenerative disease, retinal degenerative disease, angiogenic retinal disease, cancer, neurodegenerative, or autoimmune disease, and may also include other ingredients so long as the effect is not impaired below a desired level. In addition, the therapeutic or prophylactic agent of the present invention may be formulated, although the above-mentioned plant, fish, or halofuginone itself may be used as a therapeutic or prophylactic agent. The route of administration of the therapeutic or prophylactic agent of the present invention may be either oral or parenteral, and may be appropriately set according to the form of the therapeutic or prophylactic agent and the like.

For oral administration, the active ingredient may be formulated into various forms such as tablets, granules, granules, powders, capsules, and the like, and may contain additives such as binders, inclusions, excipients, lubricants, disintegrants, wetting agents, and the like, which are commonly used in the formulation. In addition, in the case of oral administration, the formulation may be formulated as a liquid state such as an internal solution, a suspension, an emulsion, or a syrup, or may be formulated as a dry state that is redissolved at the time of use.

For parenteral administration, the active ingredient may be formulated in unit dose ampoules or multi-dose containers or tubes and may also contain additives such as stabilizers, buffers, preservatives, isotonic agents, and the like.

Formulations for parenteral administration may also be formulated in powders that are redissolvable in a suitable carrier, such as sterile water, at the time of use.

Parenteral administration includes intravitreal administration, subconjunctival administration, anterior chamber administration, ocular administration, intravenous administration, intramuscular administration, subcutaneous administration, transdermal administration, intraperitoneal administration, and the like.

An appropriate administration method can be selected according to the age and symptoms of the patient. The dosage varies depending on the age, route of administration, and number of times of administration, and can be appropriately selected by a person skilled in the art.

<Hypoxia Inducer Inhibitors>

The present invention relates to plants belonging to the genus Geranium, plants belonging to the genus Morelia, plants belonging to the genus Narcissus, plants belonging to the genus Hydrangea, plants belonging to the genus Vitis, plants belonging to the genus Salix, plants belonging to the genus Camellia, plants belonging to the genus Urtica, plants belonging to the genus Cleyera, plants belonging to the genus Raphanus, plants belonging to the genus Peumus, plants belonging to the genus Passiflora, plants belonging to the genus Cryptomeria, plants belonging to the genus Tulipa, plants belonging to the genus Panax, plants belonging to the genus Curcuma, plants belonging to the genus Brassica, plants belonging to the genus Echinocactus, plants belonging to the genus Tabebuia, plants belonging to the genus Dioscorea, plants belonging to the genus Prunus, plants belonging to the genus Wolfiporia, plants belonging to the genus Amygdalus, plants belonging to the genus Spinacia, plants belonging to the genus Capsicum, plants belonging to the genus Mangifera, plants belonging to the genus Saxifraga, plants belonging to the genus Colocasia, plants belonging to the genus Ulva, plants belonging to the genus Sansevieria, plants belonging to the genus Benincasa, plants belonging to the genus Zanthoxylum, plants belonging to the genus Glycine, fishes belonging to the genus Spratelloides, and fishes belonging to the genus Hyperoglyphe, or extracts thereof.

The HIF inhibitor of the present invention preferably contains one or more selected from the group consisting of plants belonging to the genus Hydrangea, plants belonging to the genus Narcissus, plants belonging to the genus Vitis, plants belonging to the genus Brassica, and fish belonging to the genus Spratelloides, or an extract thereof.

Extracts of plants and fish which may be included in the HIF inhibitor of the present invention may be extracted in the same manner as the therapeutic or prophylactic agents described above.

The HIF inhibitor of the present invention may contain halofuginone.

The HIF inhibitors of the present invention may be formulated similarly to the treatment or prophylactic agents described above.

The mode of administration, dosage and the like are similar to the therapeutic or prophylactic agents described above.

<Retinal Neuroprotectant>

The retinal neuroprotective agent of the present invention comprises the above-mentioned HIF inhibitor.

The retinal neuroprotective agent of the present invention preferably contains halofuginone.

The mode of administration, dosage and the like are similar to the therapeutic or prophylactic agents described above.

### EMBODIMENT

<Reporter Assay>

(Establishment of an Assay System)

The HIF-activity-dependent Firefly-Luciferase and endogenous control CMV-Renilla-Luciferase were both transgenic into mouse fibroblast cell line (NIH3T3) and mouse retinal pyramidal cell line (661 W) using lentiviruses to generate stable expression lines. HIF can be stabilized by adding cobalt chloride (CoCl₂), a PHD inhibitor, to the cells, and luciferin can be added to obtain a luminescent signal correlating with HIF activity. Samples of various natural products were added to the cells to confirm the HIF inhibitory effect of the candidate on cobalt chloride-induced HIF activity.

Specifically, 1×10⁴ of NIH3T3 cells and 0.8×10⁴ of 661 W cells were seeded on a 96-well plate with the above cell lines. After cells were engrafted to the bottom of the plate, HIF-activity was induced by adding CoCl₂ at 200 μM. The luminescence strength was measured 24 hours after the CoCl₂ was added using a Dual-Glo Luciferase assay system manufactured by Promega Corporation. The candidates were added at the same time as the of the CoCl₂.

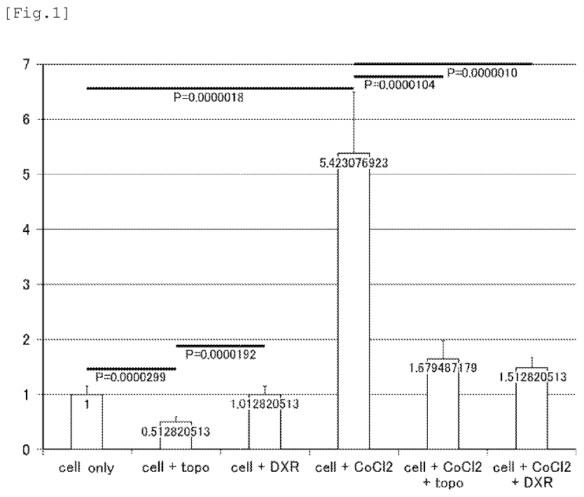

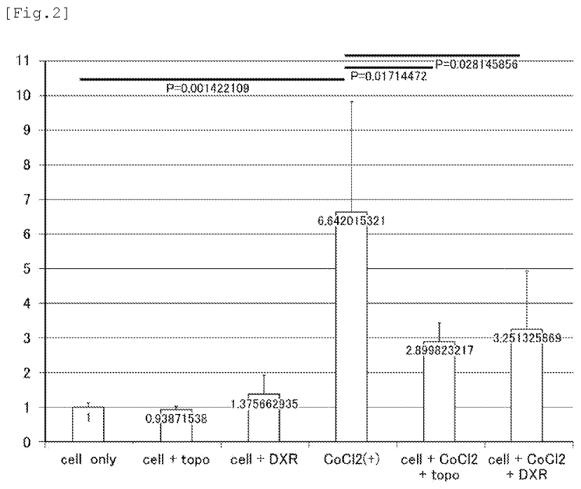

First, the activity of the activity measuring system was confirmed using topotecan and doxorubicin having HIF-inhibiting activity as positive controls. FIG. 1 shows the results for 661W and FIG. 2 shows the results for NIH3T3. In FIGS. 1 and 2, “cell” means only a cell (here, NIH3T3 or 661W), “cell+topo” means a system in which topotecan is added to the cell, “cell+DXR” means a system in which doxorubicin is added to the cell, “cell+CoCl₂” means a system in which cobalt chloride is added to the cell, “cell+CoCl₂+topo” means a system in which cobalt chloride and topotecan are added to the cell, “cell+CoCl₂+DXR” means a system in which cobalt chloride and doxorubicin are added to the cell, respectively, and this point is the same in the drawings described later.

As shown in FIGS. 1 and 2, HIF-inhibiting activity was significantly decreased against “cell+CoCl₂” in “cell+CoCl₂+topo” and “cell+CoCl₂+DXR”. In addition, since the activity of the endogenous control CMV-Renilla-Luciferase was also observed, it was confirmed that topotecan and doxorubicin exhibited HIF-inhibiting activity without cytotoxicity in this system. This assay system was used to assay the various samples below.

<Water Extract>

Various samples subjected to water extraction were extracted by the following method.

First, the sample 1 g was weighed into a 100 ml beaker, 20 ml of ultrapure water was added to the beaker, and the beaker was subjected to ultrasonic waves for 5 minutes. The sample was then placed in a stirrer bar and stirred overnight in a refrigerator at 4° C. After the stirring was completed, the sample was depressed into a 50 ml tube and centrifuged at 3000 rpm for 20 minutes at 4° C. After centrifugation, the mixture was filtered with a pleated filter paper (Miraclos part number: 475855 (manufactured by Cosmo Bio Co., Ltd.) was used), and 10 ml of ultrapure water was again added to the residue, followed by centrifugation at 3000 rpm for 20 minutes. The supernatant after centrifugation was put on a 100 ml beaker with a kimwipe and a rubber band, frozen in a freezer (−25° C.), and dried in a freeze-dryer. After drying, the weight was weighed and stored. This was an extract of the sample used in the assay with water.

<Hot Water Extraction>

Various samples subjected to hot water extraction were extracted by the following method.

First, the sample 1 g was weighed into a 100 ml beaker, 20 ml of ultrapure water was added to the beaker, and the beaker was subjected to ultrasonic waves for 5 minutes. Thereafter, the mixture was placed in a thermostat set at 95° C. (about 40 minutes) and stirred for 1 hour at 100 rpm. After the stirring was completed, the sample was depressed into a 50 ml tube and centrifuged at 3000 rpm for 20 minutes. After centrifugation, the solution was filtered with a pleated filter paper (Miraclos part number: 475855 (manufactured by Cosmo Bio Co., Ltd.) was used), 10 ml of ultrapure water was added to the residue, and centrifugation was performed at 3000 rpm for 20 minutes in the same manner as in the above-mentioned “water extraction”. The supernatant after centrifugation was put on a 100 ml beaker with a kimwipe and a rubber band, frozen in a freezer (−25° C.), and dried in a freeze-dryer. After drying, the weight was weighed and stored. This was used as a hot water extract of the sample used in the assay.

<Assay 1>

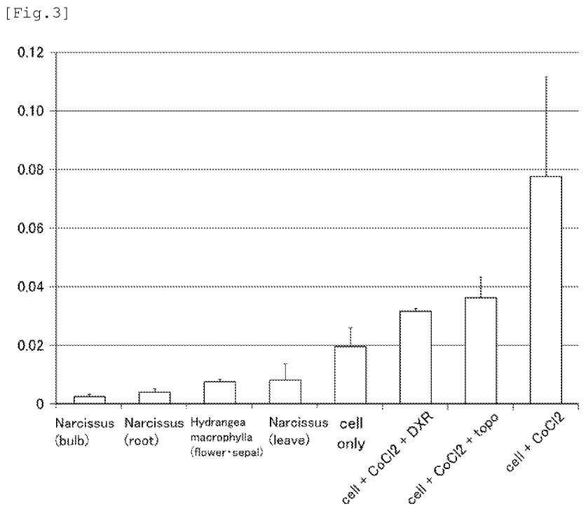

FIG. 3 shows a system in which an extract of Narcissus tazetta bulbs (hot water), N. tazetta roots (hot water), Hydrangea macrophylla flowers ⋅ calyxes (hot water) or N. tazettaleaves (hot water) are added to 661W cells with CoCl₂. Note that the notation in the above parentheses means the solvent used for extraction, and for example, the extract of N. tazetta bulbs (hot water) means the hot extract of N. tazetta bulbs.

<Assay 2>

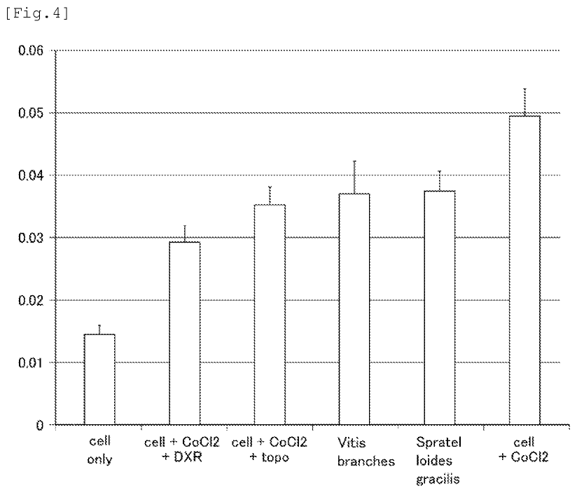

FIG. 4 shows an assay system in which extracts of Vitis vinifera branches (hot water) or Spratelloides gracilis (hot water) are added to 661W cells with CoCl₂.

<Assay 3>

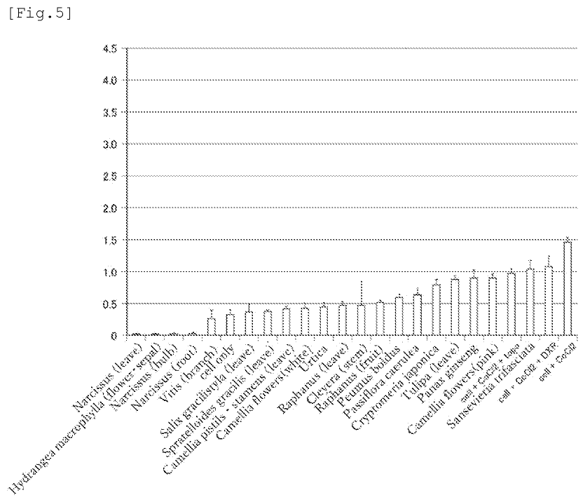

FIG. 5 shows an assay system in which extracts of Narcissus tazetta leaves (hot water), Hydrangea macrophylla flowers and calyxes (water), N. tazetta bulbs (hot water), N. tazetta roots (hot water), Vitis vinifera branches (hot water), Salix gracilistyla (hot water), Spratelloides gracilisfresh (hot water), Camellia japonica stamens and pistils (hot water), Camellia japonica flower (hot water), Urtica dioica (hot water), Raphanus sativus leaves (hot water), Cleyera japonica stems (hot water), C. japonica fruits (hot water), Peumus boldus (hot water), Passiflora caerulea (hot water), Cryptomeria japonica (hot water), Tulipa leaves (hot water), Panax ginseng (hot water), Camellia flowers (hot water), Sansevieria trifasciata (hot water) are added to NIH3T3 cells with CoCl₂.

<Assay 4>

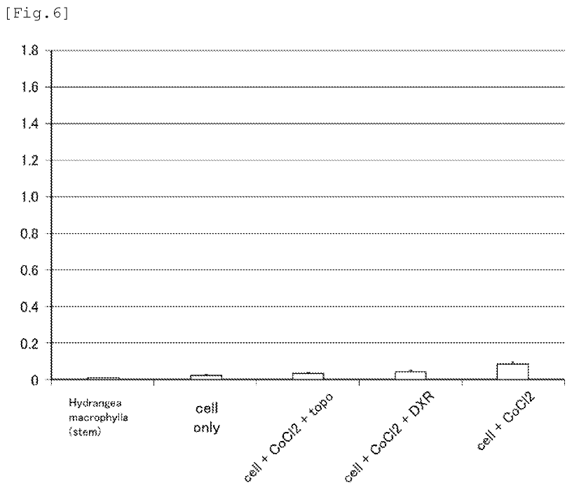

FIG. 6 shows an assay system in which am extract of Hydrangea macrophylla stems (hot water) is added to 661W cells with CoCl₂.

<Assay 5>

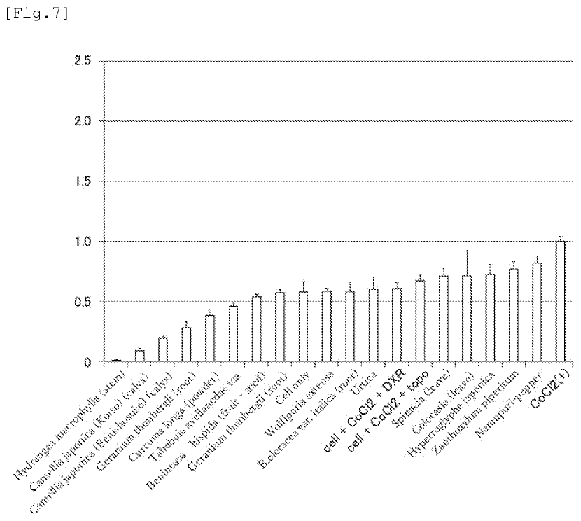

FIG. 7 shows an assay system in which extracts of Hydrangea macrophylla stems (hot water), Camellia japonica (Koiso) calyxes (water), Camellia japonica (Benishosuke) calyxes (water), Geranium thunbergii roots (water), Curcuma longa powder (water), Tabebuia avellanedae tea (hot water), Benincasa hispida fruits ⋅ seeds (water), Geranium thunbergii upper roots (water), Wolfiporia extensa (water), B. oleracea var. italica (water), Urtica (water), Spinacia leaves (water), Colocasia bulbs (water), Hyperroglyphe japonica (water), Zanthoxylum piperitum (water) or Namupuri-pepper (water) are added to NIH3T3 cells with CoCl₂.

<Assay 6>

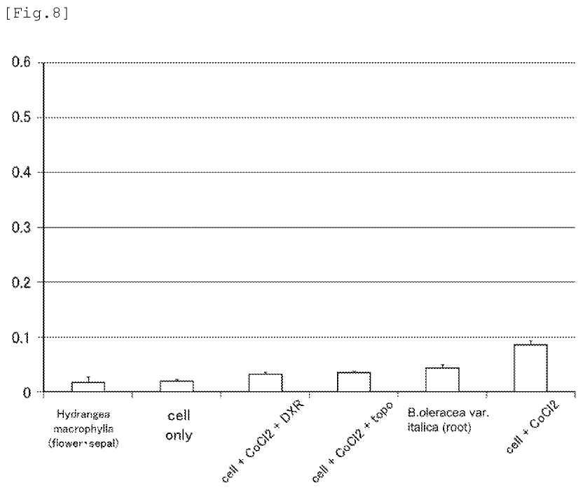

FIG. 8 shows an assay system in which extracts of Hydrangea macrophylla flowers ⋅ calyxes (hot water) or B. oleracea var. italica (hot water) are added to 661W cells with CoCl₂.

<Assay 7>

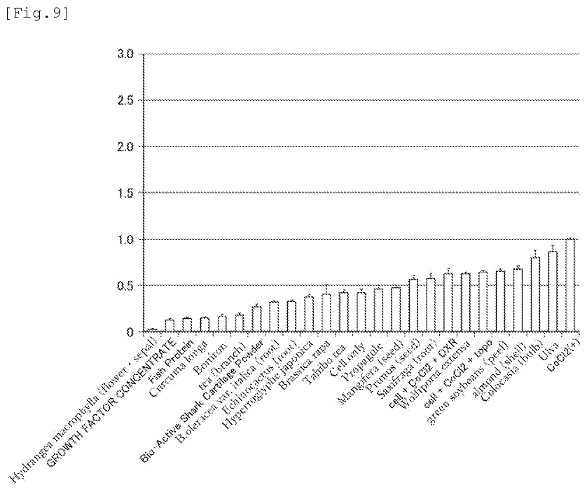

FIG. 9 shows an assay system in which extracts of Hydrangea macrophylla flowers ⋅ calyxes (water), GROWTH FACTOR CONCENTRATE (hot water), Fish Protein (hot water), Curcuma longa powder (hot water), bonilon (hot water), Camellia sinensis branches (hot water), Bio-Active Shark Cartilage Powder (hot water), B. oleracea var. italica roots (hot water), Echinocactus roots (hot water), Hyperroglyphe japonica (hot water) cacti (hot water), dashi sea bream (hot water), Tabo rapeseed (hot water), Tabo water (hot water), Mukaya (hot water), Mango and seed (water), prune and seed (hot water), Yukinoshita and root (hot water), Bukuli beans and bark (hot water), almond and shell (hot water) or Aosa and human eggsa (water) are added to the NIH3T3 cells with CoCl₂.

<Assay 8>

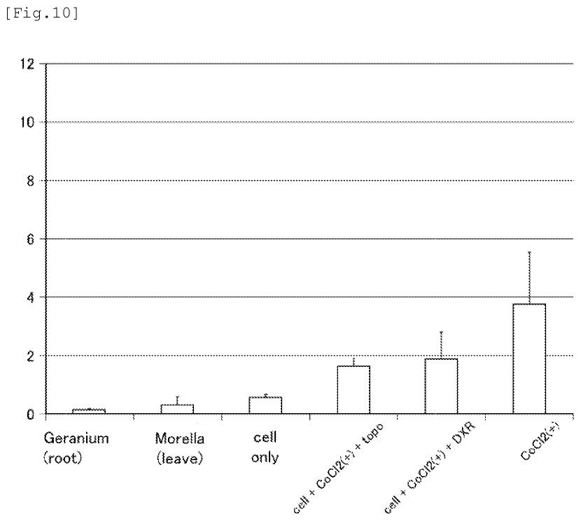

FIG. 10 shows an assay system in which extracts of Geranium thunbergii roots (hot water) or Morella rubra leaves (hot water) are added to NIH3T3 cells with CoCl₂.

Significant HIF-inhibiting activity was observed in NIH3T3 cells or 661W cells in each extract, as shown in FIGS. 3-10. In particular, the HIF inhibitory activity was observed in both NIH3T3 cells and 661W cells for Hydrangea macrophylla, Narcissus tazetta, Vitis vinifera, Spratelloides gracilis and B. oleracea var. italica, indicating that they are potent HIF inhibitors.

<Assay 9>

(Measurement of HIF Activity)

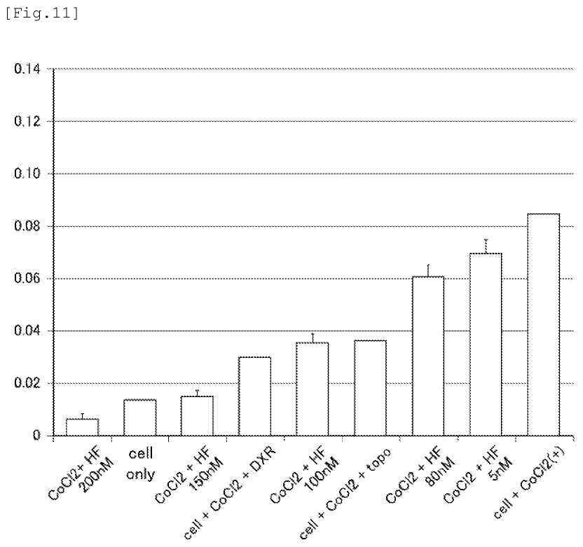

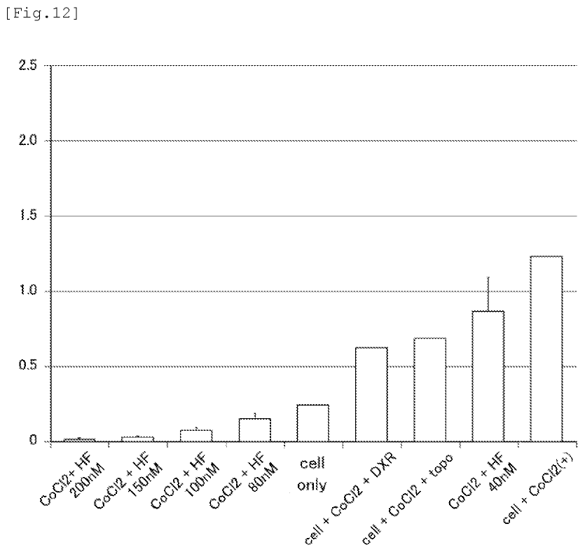

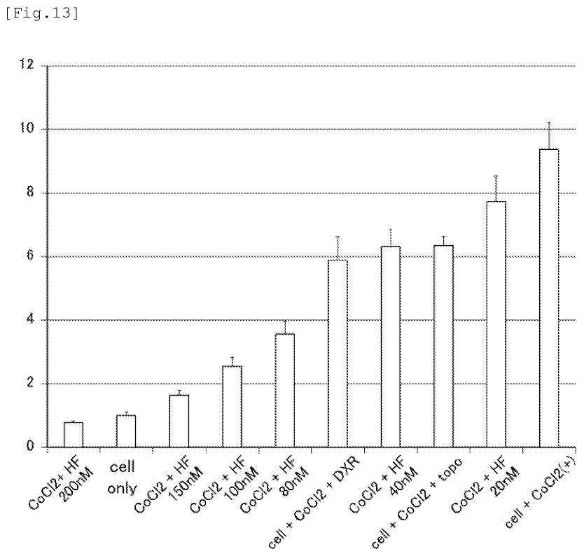

Halofuginone, a halogenated derivative of ferbrifudine in Hydrangea macrophylla, was used as a sample to measure HIF activity on 661W cells, 3T3 cells, or ARPE19 cells in the assays described above. The results are shown in FIG. 11-13. FIG. 11 shows the results for 661 W cells, FIG. 12 shows the results for NIH3T3 cells, and FIG. 13 shows the results for ARPE19 cells. In FIGS. 11 to 13, “CoCl₂+HF” indicates a system in which CoCl₂ are added to the cells. For example, “CoCl₂+HF200 nm” means that halofuginone was added to a final concentration of 200 nanometers at the time of assaying.

As shown in FIGS. 11 to 13, halofuginone exhibited significant level-dependent HIF inhibitory activity in all cell lines.

(Measurement of Gene Expression)

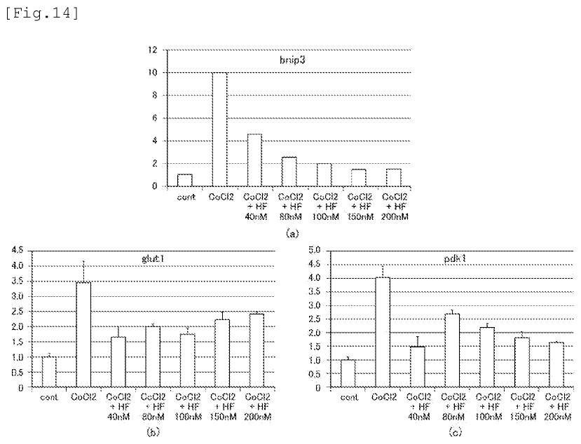

In the system of 661W cells to which the CoCl₂ was added, the expression levels of the respective genes were measured in order to examine the inhibitory effects of halofuginone on the hif gene induced by the CoCl₂ and the bnip3 gene, glut1 gene, pdk1 gene, and vegf gene which are the downstream genes. The expression level was measured by Real-time PCR. Specifically, 3×10⁴ of 661 W cells were seeded on a 24-well plate, and CoCl₂ was added at a density of 200 μM after cell immobilization. Halofuginone was added at the concentrations shown in FIG. 14 simultaneously with CoCl₂. Four hours after adding halofuginone, RNAs were extracted, cDNA was synthesized, and then Real-time PCR was performed to determine the expression levels of the respective genes. (a) The results for the bnip3 gene, (b) the glut1 gene, and (c) the pdk1 gene are shown in FIG. 14.

From the results of FIG. 14, it was found that the expression levels of the bnip gene, the glut1 gene, and the pdk1 gene were decreased by halofuginone in the 661 W-cell system. The results showed that halofuginone was effective in suppressing HIF downstream genes.

<Assay 10>

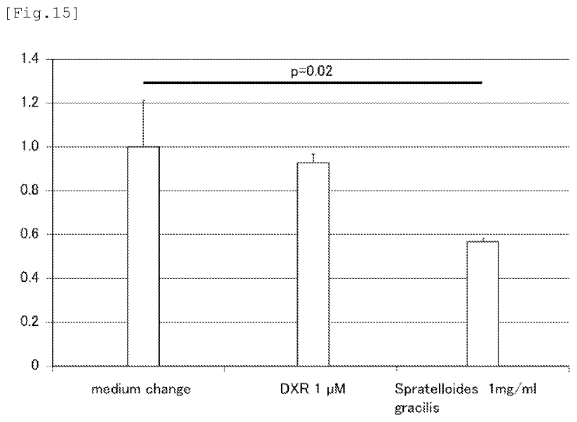

NIH3T3 cells cultured at an oxygen concentration of 5% and induced HIF activity were added at a final oxygen concentration of 1 mg/ml to perform the reporter assay described above, and the HIF activity was measured. The results are shown in FIG. 15. The term “medium change” in FIG. 15 denotes a medium that is replaced with a medium having a normal composition as a control for a medium that contains Spratelloides gracilis, and the medium is replaced with a medium having a normal composition as a control for the medium that contains mushrooms.

As shown in FIG. 15, the addition of Spratelloides gracilis showed significant HIF activity against “medium change”.

<Study of Halofuginone Administration to a Mouse Retinal Photoinjury Model>

First, 0.4 mg/kg of halofuginone solution dissolved in PBS was administered intraperitoneally once daily to BALB/c mice 8 weeks old for 5 consecutive days (halofuginone group; n=4). The control group received the same amount of PBS (phosphate buffered saline) intraperitoneally (n=4) for the same period of time.

Dark adaptation was performed on day 4 of the above period and then on day 5 a white LED of 3000 lux was irradiated for 1 hour.

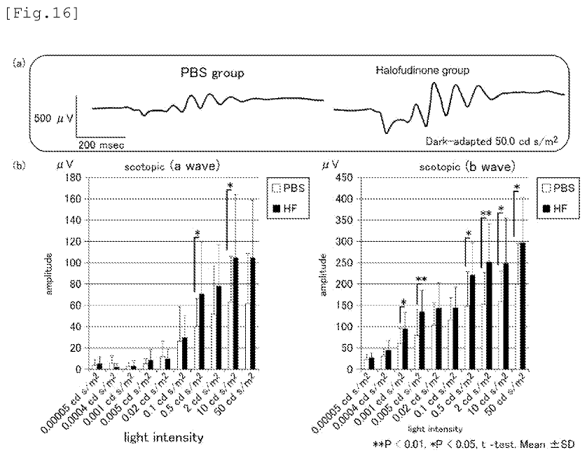

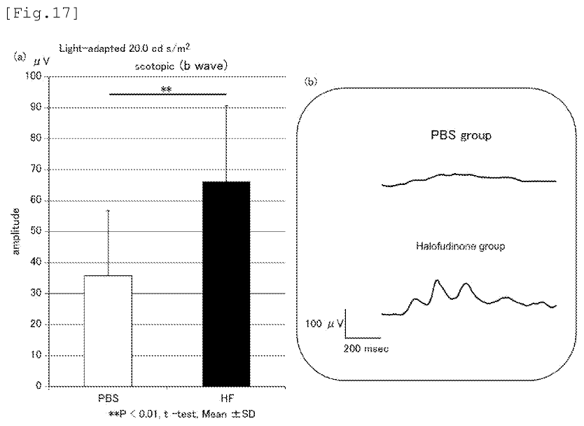

Dark adaptation was performed on day 6 after the light irradiation, and electroretinography was performed on day 7. The results are shown in FIGS. 16 and 17.

FIG. 16 is a diagram showing the results of (Dark-adapted) electroretinography (rod system and cone system function) conducted in the dark in the halofudinone and PBS groups (controls); (a) shows representative waveforms (amplitude and latency relationships) in the halofudinone and PBS groups at the light intensity 50.0 cds/m²; and (b) shows amplitude for various light intensities.

FIG. 17 is a graph showing results of electroretinography (cone system function) at a light intensity 2 of 20.0 cds/m² (Light-adapted) in the light field, and (a) shows amplitudes, and (b) shows typical waveforms (relationships between amplitudes and latencies) of the halofuginone group and the PBS-group in the light intensity.

As is clear from the results shown in FIGS. 16 and 17, the decrease in amplitude was suppressed in the halofuginone administration group compared with the PBS administration group, and the effect of suppressing retinal light disorder by the halofuginone administration was observed. Therefore, the retinal neuroprotective effect of halofuginone in the retinal photoinjury model was confirmed.

<Study of Halofuginone Administration to a Mouse Retinal Ischemia-Reperfusion Model>

The present inventors have confirmed and reported that halofuginone and the like have a neuroprotective effect using a mouse ganglion cell disorder model by vitreous administration of N-methyl-D-aspartic acid. As described above, the effect of HIF inhibitor such as halofuginone can be expected even if the lesion site in the retina is different, and therefore, it is considered that the neuroprotective effect is also exerted on the lesion in a wide range of retina such as retinal ischemia. Therefore, we tested the neuroprotective effect of halofuginone on a retinal ischemia-reperfusion model.

(Creation of Mouse Retinal Ischemia-Reperfusion Model)

First, 0.2 mg/kg of halofuginone solution dissolved in PBS was administered intraperitoneally once daily to C57BL6J mice 8 weeks old (male; manufactured by Crea Japan) for 7 consecutive days (Halofuginone group; n=4). The control group received the same dose of PBS intraperitoneally for the same period (n=4).

Retinal ischemia reperfusion treatment was then performed as follows.

Nine-week-old mice were administered intraperitoneal anesthetics (three mixtures: medetomidine 0.75 mg/kg, midazolam 4 mg/kg, and butorphanol 5 mg/kg; hereinafter referred to as “MMB”). At the same time as anesthesia, Midrin P ophthalmic solution (Santen Pharmaceutical Co., Ltd.) was used to dilate both eyes.

The physiological saline infusion bags were fixed at a height of 1.5 m from the laboratory table, inserted into the anterior chamber of the right eye of the mice with 35 G needles perfused with physiological saline, and perfusion was started. Intraocular pressure was measured every 5 minutes (Tonolab hand-held tonometer; manufactured by icare Corporation) to confirm that intraocular pressure was maintained at 85-99 mm Hg.

After 30 minutes of reflux, the needle was removed, and the left eye was treated for 30 minutes in the same manner.

For the visual evoked potentials described below, the mouse scalp was partially removed and two stainless bolts were secured to the skull at a depth of 1 mm with dental adhesive.

The bolts were fixed to the cerebral cortex at the site corresponding to the primary visual cortex (1.5 mm anterior to the lambda suture, 1.5 mm lateral to the primary visual cortex).

After the above-mentioned retinal ischemia reperfusion treatment, administration was further carried out on alternate days for 1 week, and then the following electrophysiological evaluation (retinogram and visual evoked potential) and retinal thickness evaluation were carried out.

(1) Electroretinogram (Electric Retinogram) Assessment

After 12 hours of dark adaptation, MMB anesthesia and bilateral pupillary dilation were performed. The ERGs of both eyes were measured using an LED stimulator and analysis system (PuREC; Mayo) in a stepwise manner with three stages of rods (Rod; 0.02 cds/m², 4 additions), mixing (Mix; 50 cds/m², 1), and cones (Cone; 20 cds/m², 32 additions). The amplitudes and latencies of the respective a and b waves were used for evaluation.

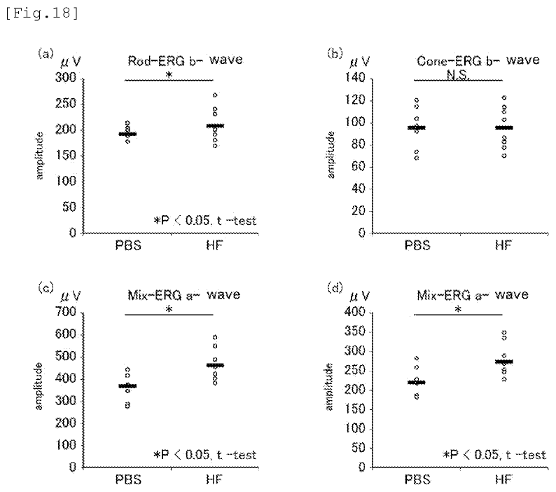

The results are shown in FIG. 18 and Table 1 below. FIG. 18 is a graph showing the results of ERG measurements in the halofuginone-treated group and the PBS-treated group, (a) shows the amplitude in the case of using the Rod ERGb wave, (b) shows the amplitude in the case of using the Cone ERGb wave, (c) shows the amplitude in the case of using the Mix ERGa wave, and (d) shows the amplitude in the case of using the Mix ERGb wave.

TABLE 1

| | | | significant |
|---|---|---|---|
| | PBS group | Halofuginone group | difference |
| | amplitude(μV) | amplitude(μV) | t test |
| Rod ERG b wave | 192 ± 13.8 | 208 ± 33.6 | p < 0.05 |
| Cone ERG bwave | 95 ± 19.5 | 95 ± 19.1 | p > 0.05 |
| Mix ERG a wave | 221 ± 38.5 | 275 ± 46.0 | p < 0.05 |
| Mix ERG b wave | 370 ± 69. 7 | 465 ± 73.8 | p < 0.05 |

As is evident from the results shown in FIG. 18 and Table 1, the halofuginone group significantly inhibited the amplitude-decrease in Rod ERG and Mix ERG compared to the PBS group, suggesting that the halofuginone administration protects retinal neurons from impairment of retinal ischemia-reperfusion.

(2) Visual Evoked Potential (Visual Evoked Potential: VEP) Assessment

The VEPs of the same individuals were then measured continuously.

Two 64 stimulus additions were performed with a 1 Hz flashing light source (3 cds/m²) with the previously placed skull bolts as positive electrodes.

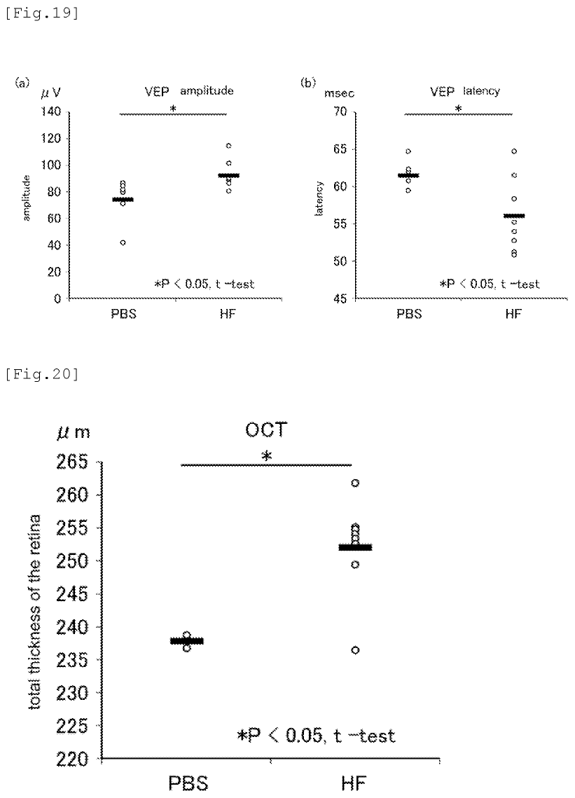

Differences (P1-N1) from each first negative crest to the next positive crest were measured as amplitudes, and latencies were also measured, and the mean of two times was evaluated as the value of one individual. The results are shown in FIG. 19.

FIG. 19(a) shows the results of the amplitude, and (b) shows the results of the latency.

As is clear from the results shown in FIG. 19, the VEP amplitude was 92±10.9 μV (t-test, p<0.05) in the halofuginone group versus 74±16.8 μV in the PBS group, and the VEP amplitude significantly decreased in the halofuginone group.

The latency of VEPs was also 56.0±5 0 m seconds (t-test, p<0.05) in the halofuginone group versus 61.4±2 0 m seconds in the PBS group, which significantly suppressed the latency prolongation in the halofuginone group. This also indicates that halofuginone has a protective effect against retinal ischemic injury.

(3) Optical Coherence Tomography (Optic Coherence Tomography) Assessment

The entire thickness of the retina was evaluated using OCTs in order to measure the total thickness of the retina in the In vivo.

Mice were fixed under MMB anesthesia and the total retinal thickness centered on the optic papilla was measured. The retinal thickness (from the inner limiting membrane to the subepithelium of retinal pigment) was measured at 10 places every 36 degrees at a radius of 250 μm from the optic nerve head using analysis software (manufactured by Bioptigen Corporation), and the mean value was evaluated as the retinal thickness of one eye. The results are shown in FIG. 20.

As is clear from the results shown in FIG. 20, the total thickness of the retina was measured by OCT to be 251.9±7.2 μm (t-test, p<0.05) in the halofuginone group versus 237.7±1.0 μm in the PBS group, indicating that the halofuginone group significantly suppressed retinal thinning caused by retinal ischemia. Histologically, this also protects retinal nerve cells from retinal damage.

1. A method of preventing and/or treating glaucoma, optic nerve disease, retinal degenerative disease, retinal pigment degeneration, or angiogenic retinal disease, in an patient, said method comprising administering as an active ingredient one or more or an extract thereof selected from plants belonging to the genus Hydrangea, plants belonging to the genus Curcuma, and fishes belonging to the genus Spratelloides or halofuginone to said patient.

2. (canceled)

3. The method according to claim 1, comprising administering as an active ingredient one or more or an extract thereof selected from the group consisting of Hydrangea macrophylla, Curcuma longa and Spratelloides gracilis or halofuginone to said patient.

4.-10. (canceled)
